(19) United States
(12) Patent Application Publication
(10) Pub. No.: US 2004/0010310 A1
(43) Pub. Date: Jan. 15, 2004

(54) Method and apparatus for correcting the refraction of an intraocular lens after implantation in the eye

(76) Inventor: Gholam A. Peyman, New Orleans, LA (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/193,140

(22) Filed: Jul. 12, 2002

(51) Int. Cl. A61F 2/16

(52) U.S. Cl. 623/6.12; 606/107; 623/920

(57) ABSTRACT

An intraocular lens, including a lens portion and a means for positioning the lens portion in the eye relative to the cornea of the eye. An outer lens surface of the lens portion is adapted to be altered after the lens portion is positioned in the eye.

### BACKGROUND OF THE INVENTION

An intraocular lens (IOL) is a transparent lens made of a synthetic or other suitable material for implantation into an eye in place of or in addition to the natural lens of the eye to correct the vision of the eye. Many different types of IOLs exist for correcting various types of vision disorders. For example, an IOL can be formed in the same shape of a natural lens of an eye that has been damaged or diseased, and can be inserted into that eye in place of the natural lens.

Alternatively, in an eye suffering from myopia, hyperopia or astigmatism, an IOL can be formed to have a bi-convex, bi-concave, plano-concave, plano-convex, concave-convex, or any other shape suitable which provides the IOL with the appropriate focusing power to correct for the error in focusing power of the eye that has the vision disorder. The suitably shaped IOL can be implanted into the eye in place of or in addition to the natural lens to thus correct the focusing power of the eye and eliminate the vision disorder.

In many conventional procedures, IOLs are used to replace the cataractous lens of a patient, with the dioptic power of the lens being calculated by a formula, which takes into consideration, the length of the eye, the position of the lens (i.e. the depth of the anterior chamber) and the power of the cornea, among other factors. Although there are several conventional formulae used for this purpose, none of the formulae achieve perfect diopter correction, and in fact many are off by about plus or minus one diopter of power.

For examples of conventional intraocular lenses see U.S. Pat. Nos. 4,666,446, 4,581,031 and 4,878,910, each to Koziol et al., U.S. Pat. No. 5,098,444 to Feaster, U.S. Pat. Nos. 5,366,502 and 5,358,520, both to Patel, and U.S. Pat. No. 4,932,971 to Kelman, as well as world patent application WO 94/07435, the disclosures of each of which are hereby incorporated herein by reference.

Recently, corneal refractive surgery, such as LASIK, has been widely used to correct various refractive errors. However, there are several risks that are involved with the correction of the refractive errors through corneal refractive surgery. For example, the wound healing after laser ablation of higher order aberrations is somewhat unpredictable, since the tissue ablation is minimal. Therefore, the unpredictable healing response can sometimes neutralize the effect of ablation by tissue under or over growth and response, reducing the chance for perfect vision. For an example of corneal refractive surgery, see U.S. Pat. No. 4,840,175 to Peyman and U.S. Pat. Nos. 4,718,418 and 4,665,913 both to L'Espersance, Jr. and U.S. Pat. No. 4669,466 to L'Espersance the entire contents of each of which are herein incorporated by reference.

Accordingly, a need exists for an IOL that can correct refractive error in the eye, whereby the process of wound healing is not a significant factor.

### SUMMARY OF THE INVENTION

Accordingly, it is an object of the present invention to provide an intraocular lens that can correct the refractive error in the eye without the need for corneal refractive surgery.

Another object of the present invention is to provide an intraocular lens that can correct the residual refractive error in the eye using a noninvasive or minimally invasive procedure.

Still another object of the present invention is to provide an intraocular lens that can have the refractive properties thereof altered after implantation in the eye.

Yet another object of the present invention is to provide an intraocular lens that can be ablated, thereby changing the refractive properties thereof, after implantation in the eye.

Still yet another object of the present invention is to provide an intraocular lens that can have its refractive properties altered using minimally invasive surgery.

Still yet another object of the present invention is to provide an intraocular lens that can have its refractive properties altered by adding or removing refractive sections.

The foregoing objects are basically attained by an intraocular lens, including a lens portion and a means for positioning the lens portion in the eye relative to the cornea of the eye. An outer lens surface is adapted to be ablated after the lens portion is positioned in the eye.

The foregoing objects are further attained by a method of changing the refractive properties of an intraocular lens. The method includes the steps of implanting the intraocular lens in the eye, the intraocular lens having a first surface and a second surface. A portion of at least one of the first and second surfaces of the intraocular lens is altered after the intraocular lens has been implanted in the eye.

The foregoing objects are further attained by a method of correcting the vision in an eye of a patient. The method includes the steps of affixing an ablatable material to an intraocular lens, positioning the intraocular lens in the eye, relative to the cornea of the eye, and ablating a portion of the ablatable material.

By forming an intraocular lens in this manner, the intraocular lens can have its refractive properties altered after it has been implanted in place of or in addition to the lens of the eye, without performing an invasive procedure.

Other objects, advantages, and salient features of the present invention will become apparent to those skilled in the art from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1 illustrates a side elevational view in cross-section of an eye with a natural lens therein;

FIG. 2 is a side elevational view in cross-section of an intraocular lens according to a preferred embodiment of the present invention;

FIG. 3 is a side elevational view in cross-section of the eye of FIG. 1, with an intraocular lens in place of the natural lens, and with a portion of the intraocular lens being ablated by a laser;

FIG. 4 is a side elevational view in cross-section of the eye of FIG. 1, with an intraocular lens positioned in the anterior chamber of the eye;

FIG. 5 is a side elevational view in cross-section of the eye of FIG. 3, after the ablation of the intraocular lens;

FIG. 6 is a front elevational view in cross-section taken through the eye directly behind the iris taken along line 6-6 in FIG. 5 to further illustrate the intraocular lens, as positioned in the eye and shown in FIG. 3;

FIG. 7 is a side elevational view in cross-section of the eye of FIG. 3, with two needles or lines inserted in the eye to irrigate and aspirate the lens and remove ablated material therefrom;

FIG. 8 is a side elevational view in cross-section of an intraocular lens according to a second embodiment of the present invention;

FIG. 9 is a side elevational view in cross-section of an intraocular lens according to a third embodiment of the present invention; and

FIG. 10 is a side elevational view in cross section of a procedure according to a fourth embodiment of the present invention.

### DETAILED DESCRIPTION OF THE INVENTION

FIG. 1 is a cross-sectional view of a normal ametropic eye 10. The eye 10 includes a cornea 12, an iris 14, a lens 16, a ciliary sulcus 18 adjacent the lens 16, a zonular ligament 20, a retina 22 and a macula 24. As illustrated, the macula 24 is located at the center of the retina 22 and is responsible for providing acute vision, such as that necessary for driving or reading.

As shown in FIG. 1, light rays 26 are focused directly on the macula 24 by the cornea 12 and lens 16. The cornea 12 has, on the average, 40 diopters of plus power, and the lens has 20 diopters of plus power (in the air). The combination of the cornea 12 and lens 16 therefore is equivalent to a very strong lens of 60 diopters. However, it is noted that when an intraocular lens having a power of plus 20 diopters is inserted into the eye, it acts as about a plus 10 diopter power lens, since the intraocular lens is positioned within the aqueous fluid in the eye. Therefore, the overall power of the eye with an intraocular lens of plus 20 diopters is about plus 50 diopters. The light rays 26 which enter the eye in a direction perpendicular or substantially perpendicular to the front surface of the cornea 12 are focused on the macula 24 and provide acute vision. The light rays 26 striking the cornea 12 obliquely are unfocused and provide peripheral, less acute vision.

FIG. 2 is side elevational view in cross-section of an intraocular lens 28 that is implantable in the eye in place of the natural lens of the eye. In this example, intraocular lens 28 has a bi-convex lens or lens portion 36 to which are attached haptics 32 and 34 that secure the intraocular lens 28 inside the eye.

As is commonly known in the art, the intraocular lens 28 is preferably a generally spherical lens, but lens 28 can have any desirable shape. For example, intraocular lens 28 can have a configuration suitable for the correction of myopia, hyperopia or astigmatism. Therefore to correct these problems in the eye, the intraocular lens can be a bi-concave lens, plano-convex lens, a concave-convex lens, a bi-concave lens, plano-concave lens, toric lens or have any other suitable shapes as known in the art.

As seen in FIG. 2, the intraocular lens preferably has a lens portion 36 that includes a main refractive portion 37 and an ablatable material or beam absorbing material 38 that surrounds and encloses the main refractive portion. The lens portion 36 has a first outer surface 40 and a second outer surface 42, which are generally defined by the ablatable material.

The main refractive portion 37 can be any shape as described above for the intraocular lens itself and is preferably formed of polymethylmethacrylate (PMMA), polymethylmethacrylate and acrylic acid, HEMA, silicone or any other suitable synthetic material which permits light to pass therethrough, i.e., it is substantially transparent. The main refractive portion has a first outer surface 44 and a second outer surface 46.

Ablatable material 38 is preferably a polymer or resin that can absorb, be ablated or be disrupted with a laser. For example, material 38 can be any ultraviolet absorbing resin, which allows light within the visible wavelength to pass therethrough, or any other synthetic material which is opaque to light in the infrared spectrum, but allows visible light to pass therethrough. Material 38 has a first inner surface 48 and a second inner surface 50.

Material 38 is preferably a separate material that is melted, glazed or mechanically or chemically affixed to the main refractive portion, using an adhesive or glue or any other type of suitable method. As shown in FIG. 2 the first inner surface 48 of material 38 can be affixed to the first outer surface 44 of the main refractive portion 37 and the second inner surface 50 can be affixed to the second inner surface 46. Furthermore, if desired, the material 38 can be affixed to only one side or a portion of the main refractive portion 37, as described in more detail below.

Additionally, the first and/or the second surfaces 44 and 46, respectively, of the main refractive portion and the rest of the refractive portion can be an ablatable material itself or a mixture of the materials disclosed for the main refractive portion and the ablatable material, and thus it would not be necessary to affix an ablatable material thereto as described above. In other words, the entire lens portion 36 could be a uniform or homogenous transparent material that could absorb, be ablated or be disrupted with a laser or a homogeneous mixture or non-homogeneous mixture of the ablatable material and the material described for the main refractive portion. For example, the intraocular lens can be made of ablatable material or a mixture of ablatable material and other polymers.

The haptics 32 and 34 can be formed using standard material, such as acrylic, silicone, polypropylene or any other suitable material and are coupled to the periphery of the lens portion 36. As seen in FIG. 2, the haptics are preferably curved and enable the intraocular lens to be positioned inside the eye and couple to a portion thereof. It is noted that the haptics described herein are only shown as a preferable embodiment, and any means known in the art for securing the intraocular lens to the eye can be used.

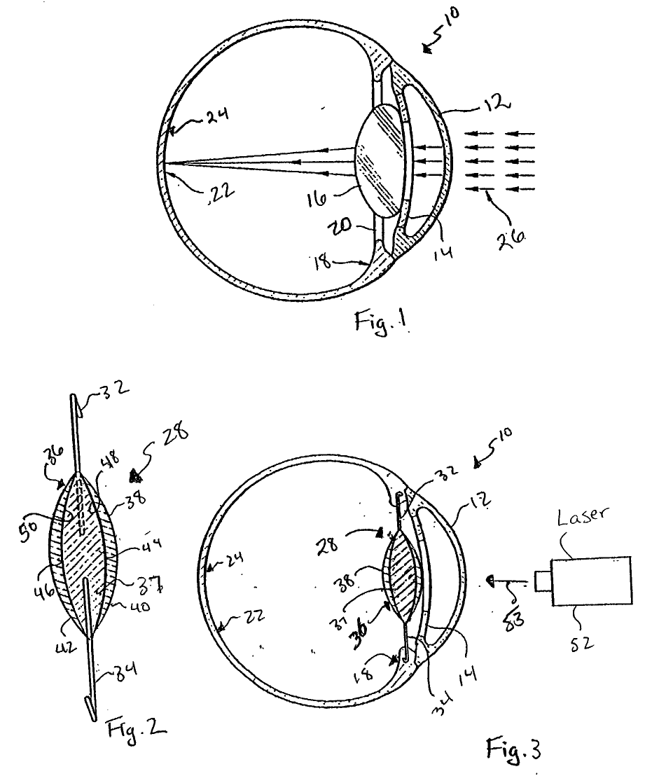

FIG. 3 is a cross-sectional view of an eye 10 into which has been mounted an intraocular lens. As known in the art, the natural lens 16 (see FIG. 1) can be removed by making an incision in the eye 10 with a microkeratome, scalpel, laser or any other suitable instrument. The natural lens 16 can then be disintegrated and removed through the incision, and the intraocular lens inserted through the incision and mounted in the eye. In this example, the intraocular lens is shown as intraocular lens 28, which includes a bi-convex lens 36. However, the intraocular lens can have any of the shapes described above. Preferably first surface 40 is positioned so that it faces in an anterior direction relative to the eye, and surface 42 is positioned so that it faces in a posterior direction relative to the eye.

As illustrated, the eye 10 includes a cornea 12, ciliary sulcus 18, retina 22 and macula 24. The lens 16 has been removed, along with all or substantially all of the zonular ligament 20 (see FIG. 1), and has been replaced with intraocular lens 28. The haptics 32 and 34 are secured to the ciliary sulcus 18 of the eye to secure the biconvex lens 36 at the appropriate location with respect to the iris 14 and cornea 12. Accordingly, the intraocular lens 28 and cornea 12 function as a lens system, which focuses light rays 36 onto the macula 24.

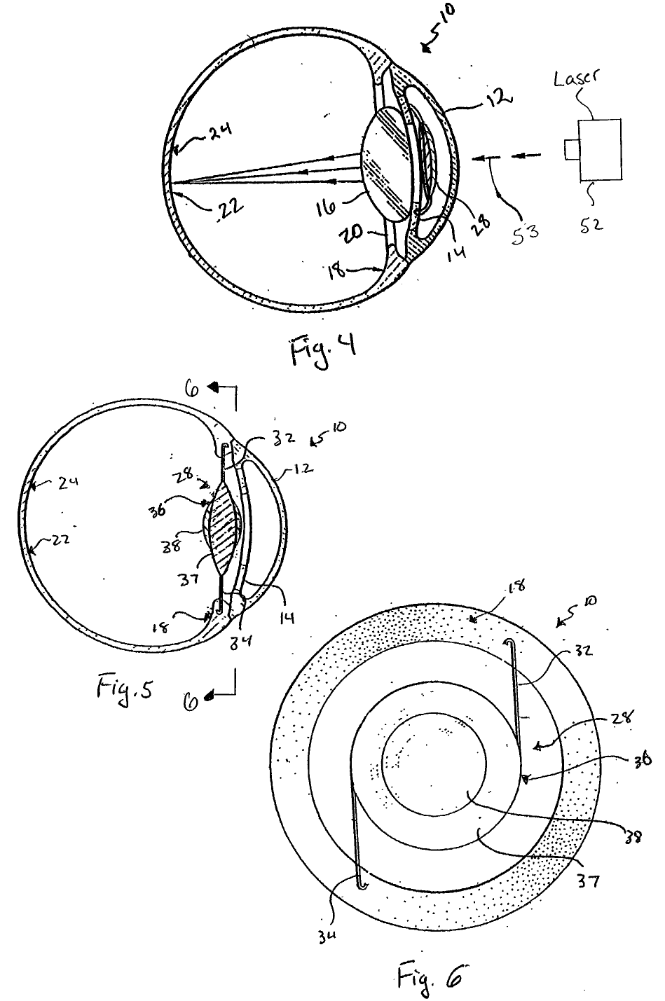

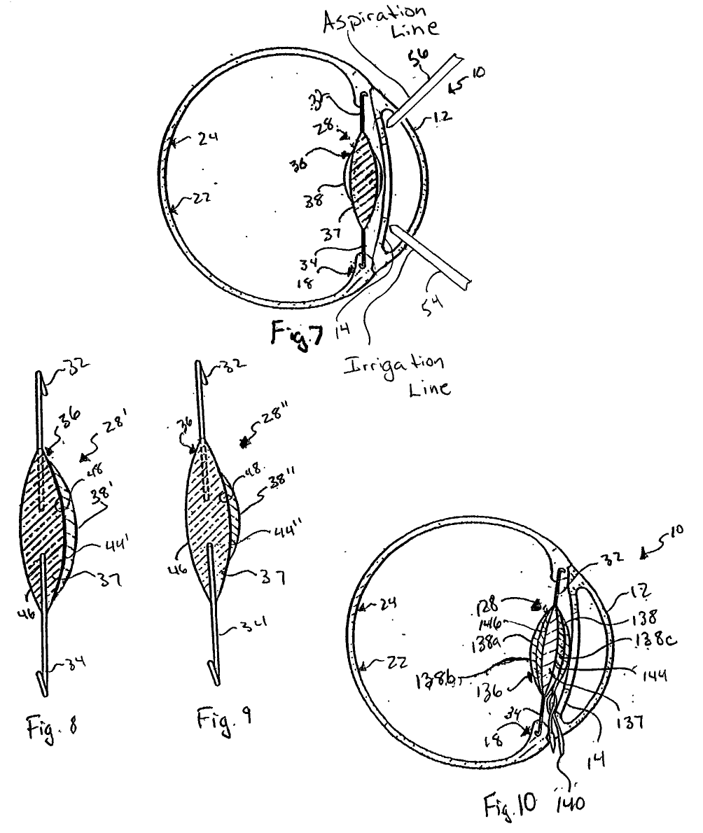

Preferably, the intraocular lens is positioned in the posterior chamber in place of natural lens 16, coupling to the ciliary sulcus 18. However, the intraocular lens 28 can be placed in the posterior chamber of the eye, in the capsular bag or couples to the ciliary sulcus, in the anterior chamber of the eye (FIG. 4), coupling to the iris or in the anterior chamber angle. Furthermore, it is not necessary in each situation to remove the natural lens, and this procedure can be used in addition to the natural lens (FIG. 4), or with a previously removed natural lens and/or an existing intraocular lens, if desired.

As is known in the art, once the intraocular lens of the present invention is in position, any wavefront and/or adoptic measuring technology can be used to determine the exact refractive index of the eye and therefore, the refractive error or any refractive aberrations of the eye. This refractive error data or information can be used by software to program a laser 52 that can ablate a portion of the intraocular lens, as described herein. Laser 52 can be any suitable laser such as an excimer laser, a continuous laser or a short pulsed laser. Preferably, if the laser is a short pulsed laser the laser can be an ultrashort or extremely short pulsed laser having a duration of about a nanosecond, a picosecond, a femtosecond or an attosecond.

As shown in FIG. 3, once the intraocular lens 28 is in the proper position, laser 52 is aimed and activated or fired at the intraocular lens 28. Preferably, laser 52 produces a beam 53 of the wavelength in the ultraviolet wavelength, such as 355 nanometers (nm). However, the beam wavelength can be between about 210 nm and about 1300 nm. This wavelength is preferably produced by a laser beam with a 1064 nm wavelength and a double frequency laser beam with a 532 nm wavelength, or any other laser or LED producing ultraviolet light, which is powerful enough that when focused on one of the surfaces of the intraocular lens it can precisely ablate a portion thereof.

Preferably, the laser 52 is focused on and ablates both surfaces 40 and 42 inside the eye to precisely correct the intraocular lens to the correct shape, as shown in FIGS. 5 and 6. For example the ablation process can be to correct or to further correct myopia, hyperopia and/or astigmatism. However, it is noted that the laser can ablate only surface 40 or surface 42, or any combination thereof to achieve the correct refractive index in the intraocular lens 28. Furthermore, the ablation process can create a bifocal lens by ablating a portion of the intraocular lens, such the bottom portion, the top portion, a side portion, or it can create one or rings around the center of the main optical axis.

Additionally, the ablation process can create one or multiple grooves, producing a prismatic change in the lens, by focal or multifocal lenses. Using this type of diffractive technology or optics, the lens can be much thinner than conventional lenses, while still producing very high refractive power. For example, a deep ablation in the center of the lens can create a minus 80 teledioptic lens. For a more detailed discussion of teledioptic lenses, see U.S. Pat. No. 6,197,057 to Peyman et al., the entire contents of which are incorporated herein by reference.

This procedure can be repeated as many times as desired to achieve the proper vision. For example, the lens can be ablated once, and the error in the eye can be remeasured using wavefront technology, and if there is still a significant amount of error, the procedure can be performed again. The subsequent measuring of the refractive properties in the eye and the subsequent procedure can be done at anytime desired, with minutes of the initial procedure, days, months or any other suitable time. It is further noted that no ablation of the eye may be necessary or desired at the initial implantation of the intraocular lens, and the ablation can be done at a later time, as described above.

Alternatively, a pulsed laser can be appropriately focused from outside of the eye onto one of the surfaces of the intraocular lens to ablate either the main refractive portion 37 or the ablatable material 38, if present. Examples of a desirable pulse lasers are an Nd-YAG laser with pulses in the nano, pico, femto or atto second duration range and having a very fine spot size, or a pulse produced by an Alexandrite laser, or any other solid, diode, dye or gas laser.

This ablation process may create tissue toxic monomers or may produce substances, which are not desirable inside the eye. However, this process is generally used to “fine tune” the intraocular lens, and therefore does not produce a significant amount of harmful material. In other words, only a minimal amount of the intraocular lens is ablated, or about a micron of thickness. This minimal amount is washed away, so that it exits the eye, using natural aqueous fluids. Furthermore, this small amount will not cause any toxicity in the body.

Nevertheless, as shown in FIG. 7, to ensure removal of all material, two small needles, lines, or devices can be inserted through a peripheral portion of the cornea. A first or irrigation needle 54 can be used with a suitable liquid reservoir and delivery system to wash the ablated material from the intraocular lens and a second or aspiration needle 56 can be used with a suitable vacuum system to remove any excess material and/or fluid. The needles are inserted into the anterior chamber, adjacent the iris, to remove the material that has flowed through or into the iris. Preferably, each needle 54 and 56 is metal or a polymer and about 1 millimeter to about 10 microns in diameter or a 32-41 gauge needle, but each needle can be any suitable size and/or material.

Each needle 54 and 56 can be inserted into separate incisions or openings in the eye, as shown in FIG. 7, or they can be inserted into the same incision or opening in the eye. Furthermore, each needle can be inserted substantially simultaneously as the other needle, or the needles can be inserted at separate times.

Second and Third Embodiments of FIGS. 8 and 9

FIGS. 8 and 9 illustrate second and third embodiments of the present intraocular lens. In each embodiment, the intraocular lenses 28′ and 28″, respectively, have ablatable material 38′ and 38″, respectively, affixed or positioned only on one side or surface of the lens. In each of FIGS. 8 and 9, the ablatable material is shown as positioned adjacent the first surface 44′ and 44″ of the main refractive portion 37′ and 37″ of each respective lens. However, the ablatable material can be positioned or affixed to either side or both sides, or the embodiments in FIGS. 8 and 9 can be mixed so that the configuration of the ablatable material in FIG. 8 can be positioned on one surface of the lens, while the other configuration is positioned on the other side of the lens.

Ablatable material 38′ is substantially similar to the configuration of ablatable material 38, in that it covers substantially all of the surface 44′, as seen in FIG. 8.

However, as noted in FIG. 9, the ablatable material 38″ only covers a portion of the surface 44″ and has a smaller diameter than both ablatable material 38 and 38′.

Both ablatable materials 38′ and 38″ are otherwise substantially similar to ablatable material 38, and any description thereof applies to the ablatable materials 38′ and 38″.

The features of intraocular lenses 28′ and 28″, which are similar to intraocular lens 28 are identified with like reference numbers. The same description of those similar features is applicable.

Fourth Embodiment of FIG. 10

Illustrated in FIG. 10, is a fourth embodiment of the present invention, which is substantially similar to the embodiments described above; however, in this embodiment, rather than ablating a portion of the intraocular lens, a section can be removed after intraocular lens 128 has been implanted in the eye. For example, intraocular lens 128 can have its refractive properties altered by inserting a piggy back lens, an additional lens or refractive section 138 or refractive sections 138a, 138b and 138c or removing one of the sections of the lens to change the refractive properties of the overall lens. Preferably, the additional refractive sections are placed or positioned adjacent to and overlying the first surface 142 and/or the second surface 144 of lens portion 136; however, the additional sections can be placed in any position in the eye desired. The sections can be removed or added using small pliers or forceps 140 via minimally invasive surgery.

The forceps are generally inserted through the opening created in the eye to insert the intraocular lens 128, as described above for intraocular lens 28. The sections 138, 138a and 138b can be any size or shape desired and can number as many or few as desired. As stated above for lens portion 36, the lens portion 136 can be any suitable shape for correcting vision in the eye. For example, lens portion 136 can be a plano-convex lens, a concave-convex lens, a bi-concave, or have any other suitable shapes as known in the art. Furthermore, sections 138, 138a and 138b can be any suitable shape to further correct or alter the refractive properties of the main refractive portion 137.

Additionally, sections 138, 138a and 138b can be ablatable material, as described above, which would allow the material to be removed via pliers or forceps and/or ablated, as described above, to alter the refractive properties of the intraocular lens.

Intraocular lens 128 is substantially similar to intraocular lens 28, and any description thereof applies to intraocular lens 128. Furthermore, the features of intraocular lens 128, which are similar to intraocular lens 28 are identified with like reference numbers. The same description of those similar features is applicable to intraocular lens 128.

While preferred embodiments have been chosen to illustrate the invention, it will be understood by those skilled in the art that various changes and modifications can be made therein without departing from the scope of the invention as defined in the appended claims.

What is claimed is:

1. A method of changing the refractive properties of an intraocular lens comprising the steps of
implanting the intraocular lens having a lens portion with a first surface and a second surface in the eye, and
altering the refractive properties of the lens portion after the intraocular lens has been implanted in the eye.

2. A method according to claim 1, further comprising the step of
positioning an ablatable material onto the lens portion.

3. A method according to claim 1, wherein
the implanting step includes implanting the intraocular lens in the anterior chamber of the eye.

4. A method according to claim 1, wherein
the implanting step includes implanting the intraocular lens in the posterior chamber of the eye.

5. A method according to claim 1, wherein
the altering step includes ablating at least one of the first and second surfaces of the lens portion with a laser.

6. A method according to claim 5, wherein
the lens portion includes an ablatable material comprising the first surface; and
the ablating step includes ablating the ablatable material with the laser.

7. A method according to claim 5, wherein
the lens portion includes an ablatable material comprising the second surface; and
the ablating step includes ablating the ablatable material with the laser.

8. A method according to claim 5, wherein
the lens portion includes an ablatable material comprising the first and second surfaces; and
the ablating step includes ablating the ablatable material on the first or second surface with the laser.

9. A method according to claim 5, wherein
the lens portion includes an ablatable material comprising the first and second surfaces; and
the ablating step includes ablating the ablatable material on the first and second surface with the laser.

10. A method according to claim 5, further comprising the step of
affixing an ablatable material to the lens portion to form the first or second surfaces; and
the ablating step includes ablating the ablatable material with the laser.

11. A method according to claim 5, wherein
said laser emits a beam of a wavelength about 355 nm to about 1300 nm.

12. A method according to claim 5, wherein
said laser is at last one of a continuous laser or a pulsed laser.

13. A method according to claim 12, wherein
said laser is a short pulse laser with a duration of at least on of the following: nanosecond, picosecond, femtosecond or attosecond.

14. A method according to claim 1, further comprising the step of
inserting an irrigation device in the eye.

15. A method according to claim 14, further comprising the step of
inserting an aspiration device in the eye.

16. A method according to claim 15, wherein
each of said irrigation and aspiration device has a diameter of about 1 mm to about 10 microns.

17. A method according to claim 1, wherein
the altering step includes implanting an additional refractive section in the eye.

18. A method according to claim 17, wherein
the altering step includes implanting the additional refractive section so that it overlies a portion of the intraocular lens.

19. A method according to claim 1, wherein
the altering step includes removing a refractive section from the intraocular lens and from the eye.

20. An intraocular lens, comprising:
a lens portion;
means for positioning said lens portion in the eye relative to the cornea of the eye; and
an outer lens portion adapted to be ablated after said lens portion is positioned in the eye.

21. An intraocular lens according to claim 20, wherein
said outer lens portion includes an ablatable material affixed to said lens portion, and adapted to be ablated by a laser.

22. An intraocular lens according to claim 21, wherein
said ablatable material is affixed on a surface of the lens portion that is positioned to face in a posterior direction relative to the eye.

23. An intraocular lens according to claim 22, wherein
said ablatable material is affixed on a surface of the lens portion that is positioned to face in an anterior direction relative to the eye.

24. An intraocular lens according to claim 21, wherein
said ablatable material is affixed on a surface of the lens portion that is positioned to face in an anterior direction relative to the eye.

25. An intraocular lens according to claim 21, wherein
said ablatable material is a synthetic polymer.

26. An intraocular lens according to claim 20, wherein
said means for positioning said lens portion includes at least one haptic.

27. A method of correcting the vision in an eye of a patient, comprising the steps of
affixing an ablatable material to an intraocular lens,
positioning the intraocular lens in the eye, relative to the cornea of the eye, and
ablating a portion of the ablatable material with a laser.

28. A method according to claim 27, wherein
the positioning step includes positioning the intraocular lens in the anterior chamber of the eye.

29. A method according to claim 27, wherein
the positioning step includes positioning the intraocular lens in the posterior chamber of the eye.

30. A method according to claim 27, wherein
the affixing step includes affixing the ablatable material on a surface of the intraocular lens that is positioned to face in a posterior direction relative to the eye.

31. A method according to claim 30, wherein
the affixing step includes affixing the ablatable material on a surface of the intraocular lens that is positioned to face in an anterior direction relative to the eye.

32. A method according to claim 27, wherein
the affixing step includes affixing the ablatable material on a surface of the intraocular lens that is positioned to face in an anterior direction relative to the eye.

33. A method according to claim 27, further comprising the step of
inserting an irrigation device in the eye.

34. A method according to claim 33, further comprising the step of
inserting an aspiration device in the eye.

35. A method according to claim 34, further comprising the step of
inserting an aspiration device in the eye.

36. A method according to claim 27, wherein
said ablating step includes ablating a portion of the ablatable material with a 355 nm wavelength laser.

37. An intraocular lens, comprising:
a lens portion;
means for positioning said lens portion in the eye relative to the cornea of the eye; and
a refractive section that is adapted to be releasably coupled to said lens portion after said lens portion has been implanted in the eye.

38. An intraocular lens according to claim 37, wherein
said refractive section overlies a first side of said lens portion.

39. An intraocular lens according to claim 37, wherein
said refractive section overlies a second side of said lens portion.

40. An intraocular lens according to claim 37, wherein
said refractive section is adapted to be removed from the eye after the intraocular lens has been implanted in the eye.

41. An intraocular lens according to claim 37, wherein
said refractive section is adapted to be inserted into the eye after the intraocular lens has been implanted in the eye.

42. An intraocular lens according to claim 37, wherein
said refractive section is formed of ablatable material.
